(19) United States
(12) Patent Application Publication
(10) Pub. No.: US 2004/0176767 A1
(43) Pub. Date: Sep. 9, 2004

(54) Fixation augmentation device and related techniques

(76) Inventor: Barry T. Bickley, North Andover, MA (US)

Correspondence Address: DALY, CROWLEY & MOFFORD, LLP, SUITE 101, 275 TURNPIKE STREET, CANTON, MA, 02021-2310, US

(21) Appl. No.: 10/246,304

(22) Filed: Sep. 18, 2002

Related U.S. Application Data
(60) Provisional application No. 60/323,347, filed on Sep. 18, 2001.
(60) Provisional application No. 60/347,212, filed on Jan. 10, 2002.
(60) Provisional application No. 60/374,534, filed on Apr. 22, 2002.

(51) Int. Cl. A61B 17/58

(52) U.S. Cl. 606/72

(57) ABSTRACT

A Fixation Augmentation Device (FAD) is presented. The FAD includes a tubular body or sleeve having a collar disposed on a first end thereof. The sleeve is provided having an outside diameter selected such that the sleeve fits into a hole drilled in a bone. The collar at the first or proximal end of the sleeve prevents advancement of the FAD into the hole with screw insertion. The outer walls of the FAD can be ribbed, threaded or fretted to enhance this fixation. Once a screw is inserted into the FAD, the shape of the walls of the FAD take advantage of the tubular nature of the bone to capture one or more cortices by virtue of the expansion of the device thereby allowing the screw and FAD to obtain purchase where a screw alone would have failed or otherwise have inadequate fixation. The FAD is also designed to collapse as it is withdrawn from a drill hole in the event of removal of the device is necessary. The FAD is designed to allow the screw to obtain secure fixation to the bone in the situations where a screw has inadequate purchase. The FAD is a relatively simple device which is relatively easy and inexpensive to manufacture and to use. Furthermore, the FAD adds no significant operative time to install.

### CROSS REFERENCE TO RELATED APPLICATIONS

This application claims priority under 35 U.S.C. §119(e) to provisional application No. 60/323,347 filed Sep. 18, 2001; to provisional application No. 60/347,212 filed Jan. 10, 2002; and to provisional application No. 60/374,534 filed Apr. 22, 2002; the disclosures of which are incorporated by reference herein.

### STATEMENT REGARDING FEDERALLY SPONSORED RESEARCH

Not Applicable

### FIELD OF THE INVENTION

The present invention relates generally to surgical fasteners. More particularly, the present invention relates to a mounting or anchoring device for use in a surgical procedure which requires insertion of a fastener in a bone or other body structure.

### BACKGROUND OF THE INVENTION

As is known in the art, fasteners such as screws or pins are commonly used to secure or mate fractured bone sections. For example, a bone fracture can be held together during the healing process by means of a fastener such as a surgical screw. Such screws and the manner in which the screws are selected for use in a surgical procedure are known.

The process of inserting the screw involves first properly aligning the bone regions to be secured by the screw. A hole is then drilled through the bone regions. After the hole is drilled, the hole is tapped. The diameter of the screw to use, the size of the hole to drill, and the size of the tap are selected in accordance with a variety of well-known factors including but not limited to the size of the bone as is generally known by those of ordinary skill in the art.

One problem which arises, however, is that as a screw is being inserted into the hole in the bone, the screw hole may become stripped and the screw no longer obtains adequate purchase in the bone. A screw having inadequate purchase is sometimes referred to as a “spinner.” Reasons for this failure of fixation include poor bone quality, over-tightening of the screw and an error in the drilling or tapping of the screw hole (e.g. the tap itself cuts a new hole because the tap does not enter the drilled hole at the proper angle, the drill does not produce a straight hole, etc . . . ). Other problems can also occur. For example, while the hole is being drilled, the fracture may shift which results in the existing hole being in a sub-optimal position.

One technique for addressing such a problems is to inject a bone cement (e.g. methylmethacrylate) into the screw hole and re-insert the screw while the bone cement hardens. This is a cumbersome and time-consuming process and also runs the risk of having the bone cement travel into the fracture site and impeding bony union. As a result of these and other risks, this technique is seldom utilized.

Another technique which is more frequently utilized than the bone cement technique is to attempt to drill another hole in a new position. However, this is not always possible due to a variety of factors including but not limited to the type and location of the bone fracture being repaired as well as the condition (e.g. quality) of the bone being repaired. A new drill hole may now create too large a drill hole or a drill hole with too thin a wall adjacent to the previous hole and again compromise or eliminate effective screw purchase. It is sometimes necessary to simply accept less than optimal fixation, or in some cases even substandard fixation, and possibly alter the post-operative rehabilitation plan. This may entail delaying weight-bearing or early motion and possibly compromising the patient's outcome.

In some cases, bone plates are used to obtain fixation and are commonly secured to the bone by screws. The use of bone plates can improve stability of the fractured bone gained by applying compression across the fracture site. Use of bone plates, however, can also make it more difficult to deal with spinners since the holes drilled in the bones must also match the locations of pre-drilled holes in the bone plates.

Another problem which arises is that osteoporotic bone or bone with abnormally thinned cortices due to failed fixation or previous infection may not allow lasting screw purchase in the bone. Thus, a surgical screw may first appear to have adequate purchase but due to bone or other conditions, the screw fails to maintain adequate screw purchase in the bone over a requisite period of time. Once the screw loosens in the bone, stability of the fractured bone gained by applying compression across the fracture site is quickly lost. A number of techniques for securing screws in a bone plate are described in U.S. Pat. Nos. 5,976,141 and 4,484,570.

Furthermore, intraoperatively bone screws frequently fail to obtain the purchase and holding power that a surgeon desires. Reasons for this failure of fixation include poor bone quality, over-tightening of the screw, an error in the drilling or tapping of the screw hole, stripping of the threads in the screw hole, etc . . . Other problems can also occur such as the fracture or implant may shift while the drill hole is created, now placing the existing hole in a sub-optimal position.

It would, therefore, be desirable to provide a device which augments secure fixation of a fastener to a bone, and to provide such a device which is easy to use, which provides reliable fixation in all types of bone or other body structures.

### SUMMARY OF THE INVENTION

In accordance with the present invention, a Fixation Augmentation Device (FAD) includes a tubular body or sleeve having a collar disposed on a first end thereof. The sleeve is provided having an outside diameter selected such that the sleeve fits into a hole drilled in a bone. The collar at the first or proximal end of the sleeve prevents advancement of the FAD into the hole with screw insertion. With this particular arrangement, a device that allows a surgeon to quickly remove a “spinner” screw and reinsert the screw in a manner such that a screw which obtains adequate purchase is provided. Furthermore by utilizing the FAD the originally selected angle of the screw relative to the fracture is maintained. The FAD improves screw purchase by effectively restoring the appropriate sized hole for screw purchase thereby giving press fit interference fixation along the inner walls of the drill hole. The outer walls of the FAD can be ribbed, threaded, fretted or otherwise textured to enhance this fixation. In addition, the shape of the outerwall of the interference portion allows the FAD to hook over the bone cortices to further enhance fixation. The FAD thus takes advantage of the tubular aspect of the bone in order to secure fixation within the bone and outside the bone. The FAD is designed to allow the screw to obtain secure fixation to the bone in the situations where a screw has inadequate purchase. The FAD is a relatively simple device which is relatively easy and inexpensive to manufacture and to use. Furthermore, the FAD adds no significant operative time to install.

In one embodiment, the FAD can be used with a bone plate. In such an embodiment, the collar portion of the FAD is provided having a size and shape which allows the sleeve portion of the FAD to be inserted into a hole in a bone through a bone plate. Thus, when the FAD is used in conjunction with a bone plate, the sleeve portion of the FAD fits through an opening in the bone plate and is inserted into a hole which has been drilled in a bone while the collar portion of the FAD fits through the opening in the bone plate but does not fit within the hole in the bone. The collar portion of the FAD can be provided having a shape which allows insertion of the FAD through the opening in the plate but which prevents rotation of the FAD during screw insertion. For example, in the case where the opening in the bone plate is provided having a round shape, the collar portion of the FAD can be provided having an oval shape. In this case, the edges of the collar would make interfering contact with the walls of the plate which define the hole to thereby prevent rotation of the FAD. This would not be accomplished with a round collar. Small fins beneath the collar can also help to accomplish this task. The tapered fins are directed radially just beneath the collar portion and cut into the proximal cortical edge. When a rotational force is applied during screw insertion these radial fins in the FAD provide a de-rotation mechanism and facilitate the passage of the screw.

In one embodiment, the sleeve portion of the FAD is tapered and the walls of the sleeve are provided having a slot provided therein. The slots extend through the entire thickness of the sleeve. The slot may be incorporated on only one sidewall of the device, or may extend through the device, thus being incorporated into the sidewall in two places. As a screw is inserted into the FAD, the slot in the sleeve portion of the FAD allows the sleeve portion of the FAD to expand thus pressing the walls of the sleeve into the bone on the inner walls of the drill hole.

In another embodiment, the FAD includes a flange at the distal end of the sleeve (i.e. at the opposite end of the sleeve from the collar). The sleeve portion of the FAD is tapered and the walls have a slot provided therein. The slots extend through the entire thickness of the sleeve. The flange portion at the distal end of the FAD hooks over the far side of the distal cortex as the screw is seated. This is accomplished by the expansion of the sleeve due to the screw entering the sleeve. The expansion can be enhanced by filling a portion of the distal end of each inner surface of the sleeve such the outer surface of the distal end of the screw pushes against the filled inner surface at the distal end of the sleeve. As the screw passes from the hollow portion of the sleeve into the filled distal end portion of the sleeve it abuts the solid inner wall which causes further expansion of the distal end and also prevents the flange from collapsing when pullout stress is applied. This approach further increases pullout strength of the FAD.

In accordance with a further aspect of the present invention, a bone fixation augmentation device, for use in a bone having a hole provided therein, includes a first collar portion adapted to mate to a first surface of a bone, an interference region projecting from the first collar portion and having a length and a width adapted to produce an interference fit between a fastener inserted in the hole and a hole wall surface. With this particular arrangement, a device for improving the purchase of a screw is provided. In one embodiment, the first collar region is disposed at a first end of the interference region and is disposed on the surface of the bone at a first end of the hole to secure the interference region in a desired location within the hole. A second, opposite end of the interference region has a second collar portion coupled thereto. The second collar portion is adapted to mate to the surface of the bone at a second, opposite end of the hole. The second collar region secures the interference region of the FAD in the hole and increases pullout strength of the FAD. In another embodiment, the FAD includes an anti-rotation structure which prevents the FAD from rotating within the hole in the bone. The anti-rotation structure can be provided as part of the first or second collars or as part of the interference region.

In still another embodiment, the interference region is provided having one or more slots therein to allow expansion of wall which comprise the interference region in response to a screw passing through the interference region.

In still another embodiment, the interference region is provided having ridges or teeth on an outer surface thereof and one or more slots therein. The slot(s) allow expansion of walls which comprise the interference region in response to a screw passing through the interference region and the ridges help secure the FAD within the bone hole.

In still another embodiment, the interference region is provided having threads on an internal surface thereof. The threads mate with screws to be inserted within the FAD.

In still another embodiment, the interference region includes wings in a portion thereof which expand in response to a screw passing through the interference region and which secure the FAD within the bone hole.

In still another embodiment, the interference region includes wings in an end portion thereof which expand in response to a screw passing through the interference region such that when the wings are deployed, the wings engage an outer surface of the bone and increase the pull force required to remove the FAD from the bone hole.

In yet another embodiment, the interference region is provided having portions thereof such that the interference region corresponds to a spring-like or clip-like structure. The interference region can also be provided having threads which mate with screws to be inserted within the FAD.

### BRIEF DESCRIPTION OF THE DRAWINGS

The invention will be more filly understood from the following detailed description taken in conjunction with the accompanying drawings, in which:

FIG. 1A is a diagram of a first embodiment of a FAD;

FIG. 1B is a cross-sectional view of the FAD of FIG. 1A;

FIG. 1C is a top view of the FAD of FIG. 1A;

FIG. 2 is a diagram of a second embodiment of a FAD;

FIG. 3A is a diagram of a third embodiment of a FAD;

FIG. 3B is a side view of the FAD of FIG. 3A;

FIG. 4A is a side view of a fourth embodiment of a FAD;

FIG. 4B is a cross-sectional view of the FAD of FIG. 4A with a screw inserted therein;

FIG. 5 is a side view of a fifth embodiment of a FAD;

FIG. 6A is a side view of a sixth embodiment of a FAD;

FIG. 6B is a side view of the FAD of FIG. 6A with a screw inserted therein;

FIG. 7A is a side view of a seventh embodiment of a FAD;

FIG. 7B is a side view of the FAD of FIG. 7A with a screw inserted therein; and

FIG. 8 is a diagram showing a tool used for insertion of a FAD into a bone.

### DETAILED DESCRIPTION OF THE INVENTION

In accordance with the present invention, a Fixation Augmentation Device (FAD) includes a tubular body or sleeve having a collar disposed on a first end thereof. The sleeve is provided having an outside diameter selected such that the sleeve fits into a hole drilled in a bone. The collar at the first or proximal end of the sleeve prevents advancement of the FAD into the hole with screw insertion. With this particular arrangement, a device that allows a surgeon to quickly remove a “spinner” screw and reinsert the screw in a manner such that a screw which obtains adequate purchase is provided.

The FAD allows a surgeon to quickly remove a “spinner” screw and reinsert the screw in a manner such that the screw obtains adequate purchase is provided. Furthermore by utilizing the FAD the originally selected angle of the screw relative to the fracture is maintained. The FAD improves screw purchase by effectively restoring the appropriate sized hole for screw purchase thereby giving press fit interference fixation along the inner walls of the drill hole. The shape of the walls of the FAD further enhance fixation by hooking over one or more cortices of the bones after screw insertion. The FAD is designed to allow the screw to obtain secure fixation to the bone in the situations where a screw has inadequate purchase. The FAD is a relatively simple device which is relatively easy and inexpensive to manufacture and to use. Furthermore, the FAD adds no significant operative time to install.

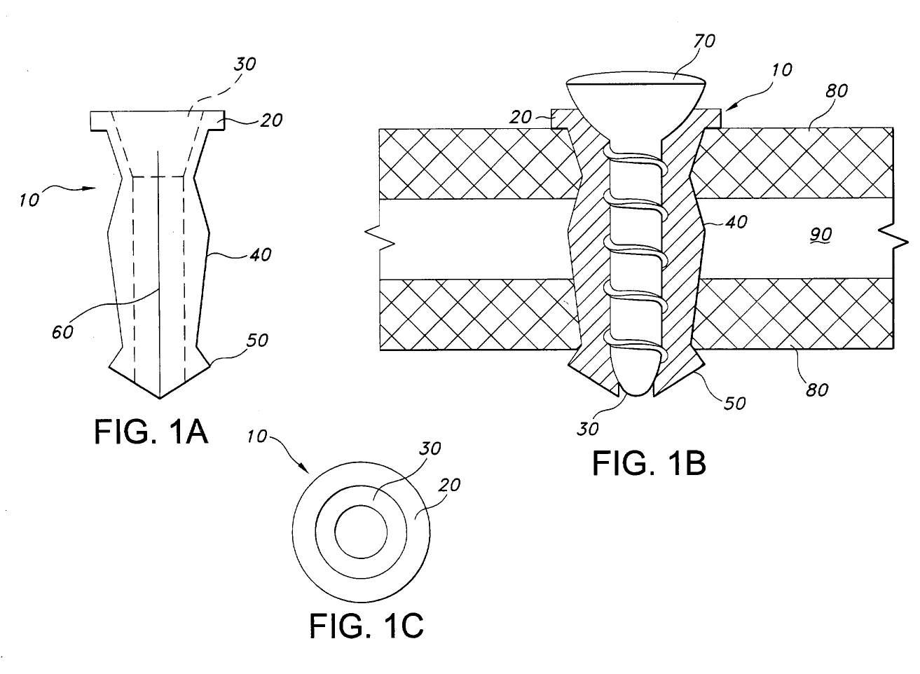

Referring now to FIGS. 1A-1C, a bone Fixation Augmentation Device (FAD) 10 includes a tubular body or sleeve 10 corresponding to an interference region. The sleeve 10 has a collar 20 disposed on a first end thereof. The sleeve 10 is provided having an outside diameter selected such that the sleeve fits into a hole drilled in a bone. The collar at the first or proximal end of the sleeve prevents advancement of the FAD into the hole with screw insertion. A first interference region 40 is provided as part of the sleeve 20. Upon insertion of a screw 70 into core 30 of the sleeve 20, the first interference region (flange) 40 expands into the hollow part 90 of the bone, providing secure fixation of the FAD to the inner cortex portions 80 of the bone. The angled sidewalls of the interference region and the flange are provided such that the FAD may be removed from the hole in the bone if desired. Removal of the screw allows the flange sidewalls and interference region sidewalls to collapse such that the FAD can be removed. A flange 50 is disposed on a second end of the sleeve 20. Upon insertion of screw 70 into core 30 of the sleeve 20, the flange 50 expands along the outer cortex 80 of the bone, providing secure fixation of the FAD to the outer cortex portion 80 of the bone. The interference region and flange can be provided having one or more slots 60 therein to allow expansion of the wall which comprises the interference region and flange in response to a screw passing through the interference region. The slots extend through the entire thickness of the sleeve. As a screw is inserted into the FAD, the slot in the sleeve portion of the FAD allows the sleeve portion of the FAD to expand thus pressing the walls of the sleeve into the bone on the inner walls of the drill hole.

The expansion of the flange portion of the sleeve can be enhanced by filling a portion of the distal end of each inner surface of the sleeve such the outer surface of the distal end of the screw pushes against the filled inner surface at the distal end of the sleeve. As the screw passes from the hollow portion of the sleeve into the filled distal-end portion of the sleeve it abuts the solid inner wall which causes further expansion of the distal end and also prevents the flange from collapsing when pullout stress is applied. This approach further increases pullout strength of the FAD.

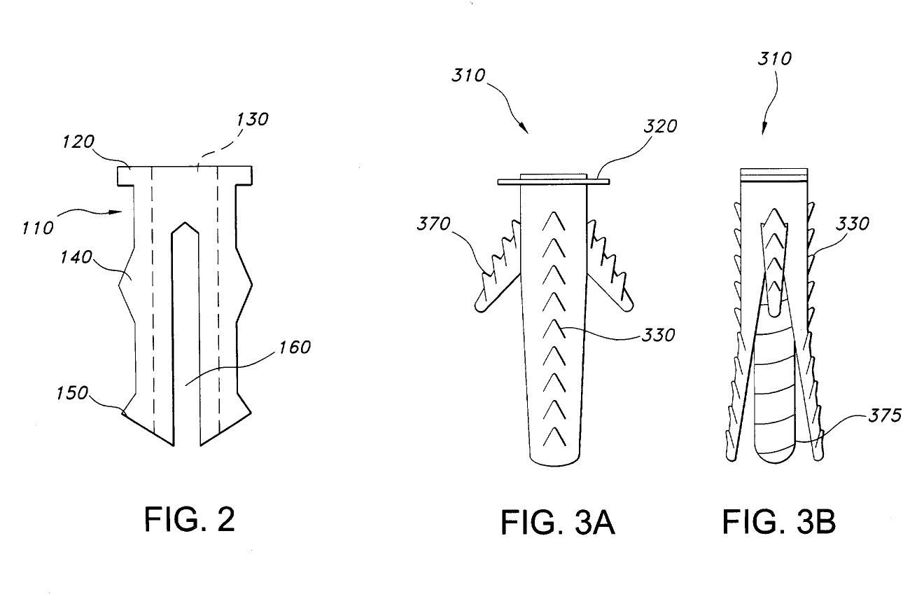

A second embodiment of a FAD 110, is shown in FIG. 2. The FAD 110 has a similar structure as FAD 10. FAD 110 includes a collar 120, a core 130, an interference region 140, a flange portion 150 and a slot 160. Operation of FAD 110 is similar to the operation of FAD 10 described above. FAD 110 clips into place in a bone hole. Slot 160 allows for the ends of the FAD to adequately clip into place.

Referring now to FIGS. 3A-B, another embodiment 310 of a FAD is shown. In this embodiment the interference region is provided having a plurality of ridges or teeth 330 on an outer surface thereof. The ridges 330 help secure the FAD within the bone hole. FAD 310 further includes wings 370 in the interference region which expand in response to a screw passing through the interference region and which secure the FAD within the bone hole. The outer walls of the FAD 310 can be ribbed, threaded or fretted to enhance the fixation of the device within the bone hole. Further the outer surface of the collar can also be ribbed, threaded or fretted to enhance the fixation of the device. As shown in to FIG. 3B, the interference region includes wings 375 in an end portion thereof which expand in response to a screw passing through the interference region such that when the wings are deployed, the wings engage an outer surface of the bone and increase the pull force required to remove the FAD 310 from the bone hole.

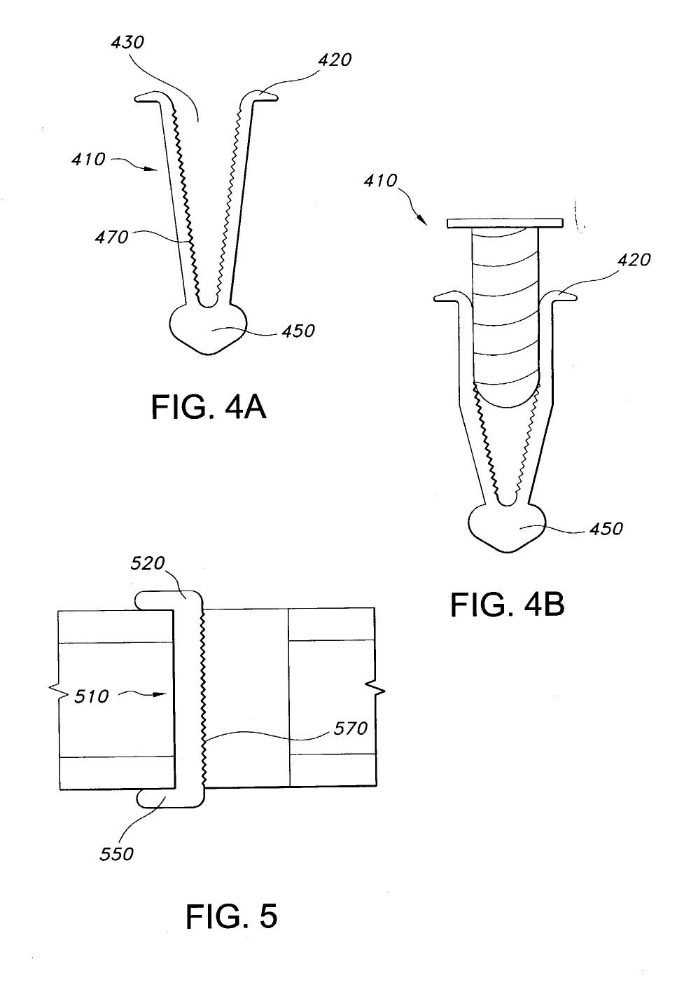

Referring now to FIGS. 4A-B, an embodiment of another Fad 410 is shown. This embodiment uses a “clip” style body having an aperture 430 disposed most of the way through. The aperture 430 includes a threaded region 470 along an inner wall. The threaded region helps secure a screw in the body to provide an expanding wedge type fit within the bone hole. A collar region 420 is disposed at a first end of the body and is disposed on the surface of the bone at a first end of the hole to secure the body in a desired location within the hole. A second, opposite end of the interference region has a second collar portion 450 coupled thereto. The second collar portion is adapted to mate to the surface of the bone at a second, opposite end of the hole. The second collar region helps secure the FAD 410 in the hole and increases pullout strength of the FAD.

FIG. 5 shows a FAD useful for smaller holes. FAD 510 is are provided from a single member which fills only a portion of a hole in a bone. FAD 510 includes collar 520 and 550 on opposing ends thereof. The FAD 510 may also have threads 570 provided therein. This FAD 510 function in a similar manner as those described above with respect to FIGS. 4A-B.

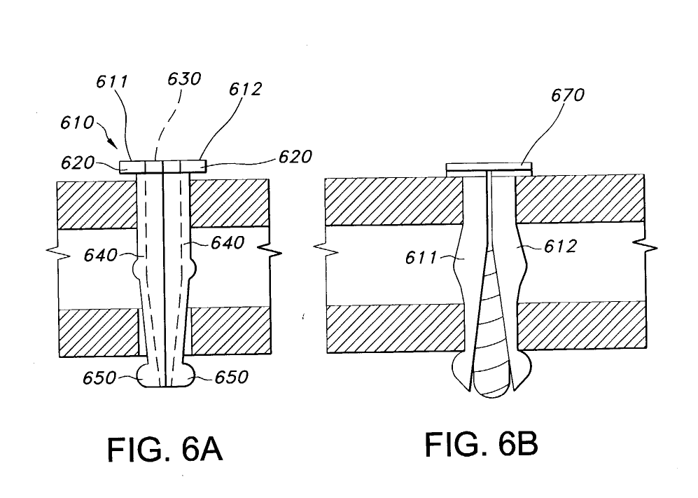

Referring now to FIGS. 6A-B, a “split” FAD 610 is shown. This FAD 610 is comprised of two complimentary pieces 611 and 612, each piece representing approximately half of the FAD. Alternately, the Fad can be comprised as a single piece which splits into two pieces upon insertion of screw 670. Each piece includes a respective collar portion 620, interference region 640 and flange region 650. The FAD is inserted into the bone hole, and upon insertion of a screw 670 the pieces 611 and 612 are separated and provide interference fit of the screw within the bone hole. The operation of the FAD 610 in other respects is similar to that of FIGS. 1A-C described above.

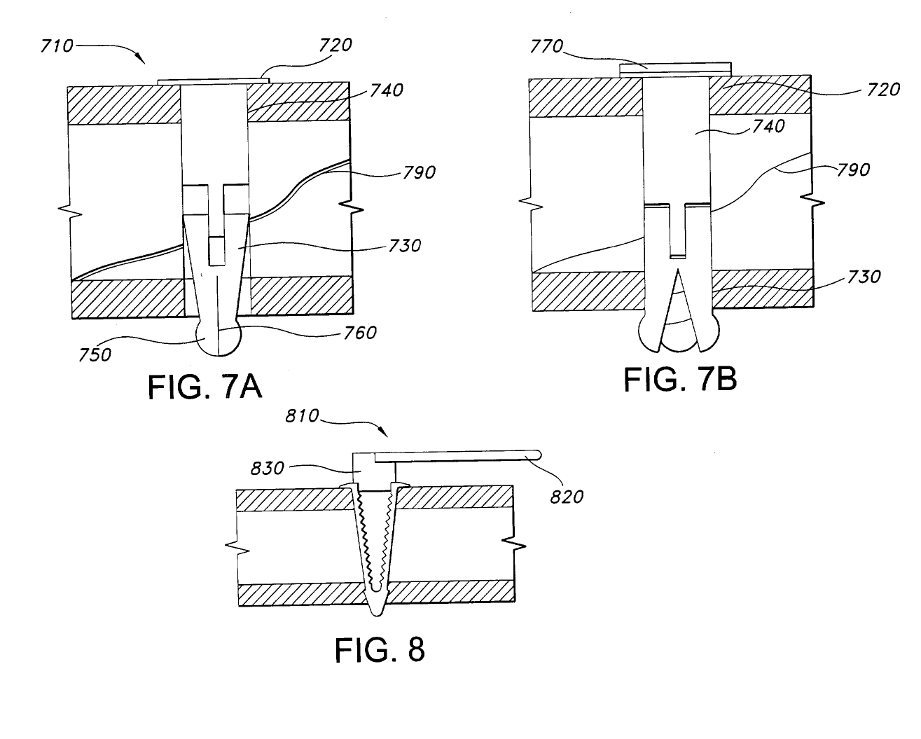

Referring now to FIGS. 7A-B, a telescoping FAD 710 is shown. This FAD 710 includes a distal portion 730 and a proximal portion 740. The two portions 730 and 740 are provided having geometries such that the portions can telescope (e.g. the distal portion can move into the proximal portion). Screw insertion pulls the distal portion 730 into the proximal portion 740 causing expansion of the proximal portion. Flared out edges of the proximal portions provide the FAD having improved fixation at the proximal cortex on the intramedullary side. A slot 760 expands upon insertion of screw 770 and flange 750 provides for secure engagement of the FAD within the bone hole. As shown in FIG. 8A, prior to engagement of the FAD 710, a fracture line 790 of a bone is uncompressed. In FIG. 8B, after insertion and engagement of the FAD 710, the fracture line 790 is compressed.

Referring now to FIG. 8, an apparatus 810 for holding a FAD during a FAD or screw insertion process includes a handle 820 and a body 830 coupled to the handle. The body 830 is adapted to hold a FAD while the FAD is inserted in a bone hole. The body holds the FAD in a manner which still allows insertion of a screw into the FAD. In one embodiment, the body is provided having a hole therein through which the screw can be inserted into the FAD. The apparatus can also include a FAD anti-rotation structure which minimizes FAD or even prevents FAD rotation which can occur when a screw is inserted into the FAD.

The procedure for FAD installation will now be described. Once the surgeon realizes that he/she has either sub-optimal fixation or a sub-optimally positioned drill hole, the hole is over drilled utilizing an appropriately sized larger drill (i.e. 4.0 mm drill for a 3.5 mm cortical screw). The FAD length corresponding to the selected screw length is then placed onto the FAD inserter and placed securely into the drill hole. Now the screw is inserted into the FAD. As the screw advances the walls of the FAD expand giving interference fixation to the bone along the inner walls of the drill hole. In addition, the expansion allows the FAD to hook over the inner side of the cortex with the flange at the central portion of the interference region. The FAD expands even further at the distal end and deploys the distal flange over the edge of the distal cortex, increasing the fixation to the bone. Once the screw is fully seated, secure fixation is once again established.

In one embodiment, the FAD can be used with a bone plate. In such an embodiment, the collar portion of the FAD is provided having a size and shape which allows the sleeve portion of the FAD to be inserted into a hole in a bone through a bone plate. Thus, when the FAD is used in conjunction with a bone plate, the sleeve portion of the FAD fits through an opening in the bone plate and is inserted into a hole which has been drilled in a bone while the collar portion of the FAD fits through the opening in the bone plate but does not fit within the hole in the bone. The collar portion of the FAD can be provided having a shape which allows insertion of the FAD through the opening in the plate but which prevents rotation of the FAD during screw insertion. For example, in the case where the opening in the bone plate is provided having a round shape, the collar portion of the FAD can be provided having an oval shape. In this case, the edges of the collar would make interfering contact with the walls of the plate which define the hole to thereby prevent rotation of the FAD. This would not be accomplished with a round collar. Small fins beneath the collar also help to accomplish this task.

Although, in the above examples the FAD collar portion is provided having an oval shape, it should be appreciated that any shape, including but not limited to rectangular, triangular, polygonal or even irregular shapes can be used. The particular shape with which to provide the collar portion of the FAD depends upon a variety of factors including but not limited to the size and shape of the opening in the bone plate, the size of the FAD, the strength required to withstand forces applied to the collar portion of the FAD and the materials from which the bone plate and FAD are manufactured.

Having described preferred embodiments of the invention it will now become apparent to those of ordinary skill in the art that other embodiments incorporating these concepts may be used. Accordingly, it is submitted that the invention should not be limited to the described embodiments but rather should be limited only by the spirit and scope of the appended claims. All publications and references cited herein are expressly incorporated herein by reference in their entirety.

What is claimed is:

1. A fixation augmentation device (FAD) comprising:
a generally tubular body having a first end and a second end, said body including an interference region located between said first end and said second end, said body insertable into a hole, said body adapted to receive a screw therein; and
a collar coaxially aligned and attached to the first end of said body.

2. The FAD of claim 1 further comprising a flange disposed at the second end of said body.

3. The FAD of claim 1 further comprising a slot extending from the second end of said body and along a portion of said body, said slot extending completely through a sidewall of said body.

4. The FAD of claim 1 further comprising a screw removably insertable into said body, said screw expanding said interference region of said body when said screw is inserted into said body.

5. The FAD of claim 1 wherein at least one of said body and said collar includes a plurality of elements disposed along an outer surface thereof, said elements selected from the group including ridges, teeth, frets, threads and texture elements.

6. The FAD of claim 1 further comprising at least one wing disposed along said body, a first end of said at least one wing extendable from said body when a screw is inserted into said body.

7. The FAD of claim 1 wherein said body has a generally tapered shape.

8. The FAD of claim 1 wherein said body comprises a left side piece and a right side piece.

9. The FAD of claim 1 wherein said body includes threads disposed along an internal surface thereof.

10. A fixation augmentation device (FAD) comprising:
a clip shaped body having a first end and a second end, said body insertable into a hole; and
a collar coaxially aligned and attached to the first end of said body.

11. The FAD of claim 10 further comprising a flange disposed at the second end of said body.

12. The FAD of claim 10 further comprising a screw, said body providing for fixation of said screw within the hole.

13. The FAD of claim 10 wherein at least one of said body and said collar includes a plurality of elements disposed along an outer surface thereof, said elements selected from the group including ridges, teeth, frets and threads.

14. The FAD of claim 10 wherein said body includes threads disposed along an internal surface thereof.

15. A fixation augmentation device (FAD) comprising:
a first generally tubular body piece having a first end and a second end, said first body piece body including an interference region located between said first end and said second end, said first body piece insertable into a hole, said first body piece adapted to receive a screw therein;
a second generally tubular body piece having a first end and a second end, said second body piece insertable into a hole, said second body piece adapted to receive a screw therein, the first end of said second body receivable by said second end of said first body piece when a screw is inserted in said first body piece and said second body piece;
a collar coaxially aligned and attached to the first end of said first body piece; and
a flange disposed at the second end of said second body piece.

16. The FAD of claim 15 further comprising a slot extending from the second end of said second body piece and along a portion of said second body piece, said slot extending completely through a sidewall of said second body piece.

17. The FAD of claim 15 further comprising a screw removably insertable into said first body piece and said second body piece, said screw expanding said interference region of said first body piece when said screw is inserted into said first body piece, said screw expanding said flange when said screw is inserted into said second body piece.

18. The FAD of claim 15 wherein said first body piece includes threads disposed along an internal surface thereof.

19. The FAD of claim 15 wherein said second body piece includes threads disposed along an internal surface thereof.
